(19) United States
(12) Patent Application Publication
(10) Pub. No.: US 2003/0215864 A1
(43) Pub. Date: Nov. 20, 2003

(54) Compositions and methods related to two-arm nucleic acid probes

(75) Inventors: Rudolf Gilmanshin, Waltham, MA (US); Nuno Goncalves, Somerville, MA (US)

Correspondence Address: Maria A. Trevisan, Wolf, Greenfield & Sacks, P.C., 600 Atlantic Avenue, Boston, MA, 02210, US

(73) Assignee: U.S. Genomics, Inc., Woburn, MA (US)

(21) Appl. No.: 10/421,644

(22) Filed: Apr. 23, 2003

Related U.S. Application Data
(60) Provisional application No. 60/374,749, filed on Apr. 23, 2002.

(51) Int. Cl. C12Q 1/68; C07H 21/04; C07K 14/00

(52) U.S. Cl. 435/6; 530/350; 536/23.1

(57) ABSTRACT

The invention provides compositions and methods of use relating to nucleic acid detection probes that comprise a Hoogsteen binding arm and a Watson-Crick binding arm that bind to adjacent but not identical target sites.

### RELATED APPLICATIONS

This application claims priority to U.S. Provisional Patent Application filed Apr. 23, 2002, entitled “NOVEL TWO-ARM PNA DESIGN FOR EXPANDED TARGETING OF DNA MOTIFS USING WATSON-CRICK AND HOOGSTEEN BINDING STRANDS BINDING TO DIFFERENT PORTIONS OF THE MOTIF”, Serial No. 60/374,749, the contents of which are incorporated by reference herein in their entirety.

### FIELD OF THE INVENTION

The invention relates to a novel molecule that is suitable for use as a probe for nucleic acid molecules.

### BACKGROUND OF THE INVENTION

Nucleic acid molecules such as DNA and RNA and nucleic acid mimics such as peptide nucleic acids (PNAs) or locked nucleic acids (LNAs) have been used as probes. Examples of PNA probes include single-stranded PNA (ssPNA) probes, bisPNA probes, and pseudocomplementary PNA (pcPNA) probes.

ssPNA binds to single-stranded DNA (ssDNA) in different modes. Depending upon its sequence (and conversely that of its target), ssPNA can form a Watson-Crick PNA/DNA hybrid of a PNA/DNA/PNA triplex where on PNA strand binds by a Watson-Crick mechanism and the other binds by a Hoogsteen mechanism. (Wittung et al. (1997) Biochemistry, 36: 7973-7979; Kosaganov et al. (2000) Biochemistry 39: 11742-11747).

ssPNA binds to double-stranded (dsDNA) either by a Watson-Crick or a Hoogsteen bonding mechanism. In the former case, one of the DNA stands is displaced and ssPNA takes it place as the complementary strand. In the latter case, ssPNA forms a PNA/DNA/DNA triplex via Hoogsteen hybridization without disturbing the dsDNA structure. Triplex formation resulting from Hoogsteen hybridization has sequence limitations since only a sufficiently long polypurine target sequence will be bound by the ssPNA (Sinden, 1994). Consequently, the ssPNA can have either a polypurine or polypyrimidine sequence.

At high concentration of ssPNA, the rate limiting step for its hybridization to dsDNA using Watson-Crick base pairing is the local melting (i.e., opening) of the double-stranded region of the target. This process has a high energetic barrier and is therefore slow. It can, however, be enhanced by increasing temperature. Since local melting is rare and randomly spaced along a target nucleic acid or sequence, particularly at room temperature, the ssPNA must be located in close proximity to its target site in order to enter and hybridize to its target efficiently. To increase the probability that a ssPNA will be in the vicinity of a target site on the nucleic acid molecule at the time of local melting, either the concentration of ssPNA can be increased or positive charges can be included in the ssPNA structure to increase local ssPNA concentration in vicinity of the nucleic acid molecule (Kosaganov et al., 2000).

FIGS. 1A-1D illustrate the different modes of binding and complex formation between a target DNA and probes of varying types. ssPNAs binding in either a Watson-Crick or Hoogsteen manner to ssDNA or dsDNA are shown in FIG. 1A and FIG. 1B. In a Watson-Crick hybrid, the PNA C-terminus is aligned with the 5′ terminus of the DNA. In a Hoogsteen hybrid, the PNA N-terminus is aligned with the 5′ terminus of the DNA. Hoogsteen binding imposes certain requirements on the target site (and thus the ssPNA sequence), and orientation of the ssPNA in a Hoogsteen hybrid will depend on its sequence, as shown in the FIG. 1C. In FIG. 1C, the target site is bound to the top ssPNA by Hoogsteen pairing, and by the bottom ssPNA by Watson-Crick pairing. The use of two PNAs can lead to a ssPNA/ssDNA/ssPNA triplex as illustrated in FIGS. 1C and 1D. By connecting the ssPNA to each other a bisPNA is formed, as shown in FIG. 1D, and this hybridizes faster and forms more stable complexes with the target DNA due to the increased amount of base pairing, relative to the individual ssPNAs.

PNA/DNA/PNA triplexes are also possible if two ssPNAs with complementary sequences are used to bind to the same target sequence. When connected by a linker, the two ssPNAs are referred to as bisPNA. A bisPNA is capable of stable complex formation even with relatively short targets because two PNA base pairs are formed with every base of the target nucleic acid molecule. Moreover, they have relatively fast hybridization rates due to the presence of the Hoogsteen strand on bisPNA which does not require local melting of a double-stranded nucleic acid in order to bind and concentrates the PNA to the target site allowing for a faster Watson-Crick reaction. The process therefore has a lower energy barrier and proceeds more quickly than ssPNA. However, as with the Hoogsteen binding of ssPNA, there still exists a target sequence limitation. BisPNA binding to ssDNA is shown in FIG. 1D.

Pseudo-complementary PNAs (pcPNAs) can bind to any target having at least 33% adenine or thymidine residues in its sequence. (Izvolsky et al., 2000) These PNAs invade dsDNA and bind both displaced strands in a Watson-Crick manner. Their rate of binding is slow and inefficient since they lack a Hoogsteen binding element.

### SUMMARY OF THE INVENTION

The invention relates in part to the discovery of a new molecule that is capable of binding to a target nucleic acid molecule using both Hoogsteen base pairing and Watson-Crick base pairing. This novel molecule is referred to as a two-arm probe, as it is comprised of two strands or “arms”, one which is capable of Hoogsteen binding and one which is capable of Watson-Crick binding. The two arms referred to herein as the ‘Hoogsteen binding strand’ or ‘Hoogsteen binding arm’ and the ‘Watson-Crick binding strand’ or ‘Watson-Crick binding arm’, do not necessarily bind to the same site on a target nucleic acid. Rather they bind to different sequences that are either cis or trans relative to each other depending on the composition of the Hoogsteen binding arm. Cis sites are sites that are located on the same strand of target and may be contiguous with each other, although there may be a certain amount of distance between them. Trans sites are sites that are located on opposite strands of a double-stranded target. The Watson-Crick and Hoogsteen binding arms of the two-arm probes can be made from nucleic acid molecules such as DNA or RNA, or from nucleic acid mimics such as PNAs (e.g., ssPNA, pcPNA, and the like), and LNAs, among others. In some important embodiments, one or both arms are PNAs.

BisPNAs bind to nucleic acid molecules using both Hoogsteen and Watson-Crick binding, although the “arms” of a bisPNA must necessarily bind to the same site on a target nucleic acid molecule. Moreover, because the Hoogsteen binding arm of a bisPNA can generally only bind to polypurine stretches of nucleic acid sequence, the number and diversity of sequences that can be detected using purely bisPNAs is somewhat limited.

The invention, on the other hand, provides a molecule having the advantages of bisPNA molecules, but capable of identifying unique sequences due to the presence of the Watson-Crick binding arm. That is, the two-arm probes of the invention bind to a subset of the target nucleic acid molecules that are bound by a typical bisPNA, and their binding pattern is determined in part by the Watson-Crick binding arm sequence.

Generally, the Hoogsteen binding arm of this new type of probe binds to polypurine target sites, although it may itself be comprised of a polypurine or a polypyrimidine nucleotide sequence. The Watson-Crick binding arm of the new probe can bind to any nucleotide sequence to which it is complementary. Accordingly, much of the sequence diversity derives from the Watson-Crick binding arm of the two-arm probe. Two-arm probes therefore will bind to rarer sequences than will bisPNAs, but will still retain the binding efficiency of bisPNAs. Although a Hoogsteen complex such as that formed with a bisPNA is dependent upon a minimal length (in order to exist at the incubation temperature for a specified time), the two-arm probes described herein can be further designed to include polypurine Hoogsteen binding arms or to be shorter than the Hoogsteen binding arms of a bisPNA because binding stability is imparted by the Watson-Crick binding arm as well.

Thus, in one aspect the invention provides a composition comprising a two-arm probe. The composition more specifically comprises a Hoogsteen binding arm that binds by Hoogsteen base pairing to a target nucleic acid molecule at a first target site, and a Watson-Crick binding arm that binds by Watson-Crick base pairing to the target nucleic acid molecule at a second target site. The Hoogsteen binding arm and the Watson-Crick binding arm are conjugated to each other.

The Hoogsteen binding arm and Watson-Crick binding arm are each a polymer, preferably a linear polymer, comprising nucleic acid residues (e.g., nucleotides, nucleosides, or organic bases such as adenine, thymine, uracil, cytosine, guanine, or inosine), or mimics of nucleic acid residues. The polymer backbone may be any backbone that links the nucleic acid residues (or mimics thereof) together, and therefore may be a phosphodiester backbone, a phosphorothioate backbone, a peptide backbone, and the like. The arms do not have to be homogeneous in composition but rather each may contain a combination of nucleic acid residues and nucleic acid residue mimics, as well as a combination of backbone linkages such as a combination of phosphodiester linkages and peptide linkages, as an example. Accordingly, each of the arms may be comprised of nucleic acid or nucleic acid mimic elements, such as those described herein.

The Hoogsteen and Watson-Crick binding arms may be comprised in part or in their entirety of DNA, RNA, PNA or LNA, mimics thereof, and combinations of the foregoing. Preferably at least one, and more preferably both arms are comprised of PNA. The Hoogsteen binding arm and/or the Watson-Crick binding arm may each independently have at least one backbone modification. The backbone modification of one arm may be different from that of the other arm. In some embodiments, the backbone modification is a peptide modification (such as in a PNA) or a phosphorothioate modification, but it is not so limited.

The Hoogsteen binding arm and the Watson-Crick binding arm are conjugated to each other, for example either covalently or non-covalently. In some embodiments, they are conjugated to each other using a linker molecule (which also may be referred to herein as a tether). The linker molecule may be any linker suitable to conjugated the arms to each other without impacting upon their ability to bind to their respective target sites on a target nucleic acid molecule. They include but are not limited to 8-amino-3,6-dioxaoctanoic acid (O-linker), E-linker, and X-linker. In some instances, the linker molecule comprises a cleavable bond, preferably a readily cleavable bond such as a bond that is cleaved upon exposure to an external stimulus such as light (perhaps of a particular wavelength) or a chemical reagent. The linker molecule may be any length, depending on the application for which the two-arm probes is used. In some embodiments, it has a length of less than 100 Angstroms, less than 75 Angstroms, less than 50 Angstroms, less than 25 Angstroms, or less than 10 Angstroms.

The Hoogsteen binding arm has a nucleotide sequence that is a homopurine nucleotide sequence or homopyrimidine nucleotide sequence. As used herein, the term “nucleotide sequence refers to the sequence of bases on each unit of the polymer that makes up an arm of the probe. Accordingly, in some instances, the “nucleotides” as used herein will lack a sugar and possibly a phosphate residue, but will still comprise the organic base involved in base pairing with a complementary strand. This may be the case, for example, when the arm contains one or more PNA residues. The same proviso applies for the Watson-Crick binding arm, which itself may have a nucleotide sequence that is random.

Either or both the Hoogsteen binding arm and the Watson-Crick binding arm may be any length, depending upon the application, and may range from 2 to more than 1000 nucleotides in length, more preferably from 2 to 100 nucleotides in length and even more preferably between 2-20 nucleotides in length. In one embodiment, the arms are independently 5-12 nucleotides in length. The length of one arm is independent of the length of the other arm, and hence the lengths of the Hoogsteen and Watson-Crick binding arms may be the same or they may be different.

In one embodiment, the first target site and the second target site are spaced apart from each other (on the target nucleic acid molecule, which may be a single-stranded or a double-stranded nucleic acid molecule) by a distance of 1 base pair, 2 base pairs, 5 base pairs, 7 base pairs, 10 base pairs, 20 base pairs, and 25 base pairs, or more, depending upon the application and sequence resolution desired. In other embodiments, the distance is 0-100 bp, or 3-15 bp. In some related embodiments, the Hoogsteen binding arm and the Watson-Crick binding arm are spaced apart from each other by a distance of 1 base pair, 2 base pairs, 5 base pairs, 7 base pairs, 10 base pairs, 20 base pairs, 25 base pairs, or more. Other embodiments, the distance is 0-100 bp, or 3-15 bp. Distances in base pairs can be converted into Angstrom distances by one of ordinary skill in the art. This distance may correspond to the distance between the connected ends of the Hoogsteen binding arm and the Watson-Crick binding arm. For example, if both arms were PNAs such that both had carboxy (C) and amino (N) termini, then this distance would correspond to the distance between the N-terminus of the Hoogsteen binding arm and the C-terminus of the Watson-Crick binding arm (for example as shown in FIG. 3A). This distance may also correspond to the distance between these ends when both arms are bound to their target sites.

In some embodiments, the Hoogsteen binding arm is conjugated to an agent and/or the Watson-Crick binding arm is conjugated to an agent. The agent may be a detectable label.

The two-arm probe (and/or its individual arm constituents) can include a detectable label selected from the group including but not limited to an electron spin resonance molecule (e.g., nitroxyl radicals), a fluorescent molecule, a chemiluminescent molecule, a radioisotope, an enzyme substrate, a biotin molecule, an avidin molecule, an electrical charge transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, a nucleic acid, a carbohydrate, an antigen, a hapten, an antibody, an antibody fragment, and a lipid.

The detectable label can be detected using a detection system. The detection system may be electrical in nature (such as a charge coupled device (CCD) detection system) or it may be non-electrical in nature (such as a photographic film detection system), but is not so limited. The detection system may be selected from the group including but not limited to a charge coupled device detection system, an electron spin resonance detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system.

The agent may also be a cytotoxic agent or a nucleic acid cleaving agent, but it is not so limited.

The target nucleic acid molecule may be a DNA or an RNA, such as genomic DNA, mitochondrial DNA, cDNA, mRNA, or rRNA, but it is not so limited. The target nucleic acid molecule may also be labeled with an agent such as a detectable label. These detectable labels may label the backbone of the target nucleic acid molecule (in whole or in part), or it may label specific “landmarks” on the target nucleic acid molecule (such as centromeres or repetitive sequences).

In a related aspect, the invention provides a two-arm probe such as that disclosed above, and including a linker that conjugates the Hoogsteen binding arm to the Watson-Crick binding arm.

In still another aspect, the invention provides a method for labeling a target nucleic acid molecule comprising contacting the target nucleic acid molecule with a two-arm probe composition such as that disclosed above, and allowing the composition to bind specifically to the target nucleic acid molecule.

The embodiments recited above for the two-arm probe composition apply equally to this method, and therefore will not be repeated herein.

The method may further comprise additional steps such as but not limited to detecting binding of the two-arm probe to the target nucleic acid molecule, or determining a pattern of binding of the two-arm probe to the target nucleic acid molecule. Binding of the two-arm probe to the target nucleic acid molecule may be determined using a linear polymer analysis system such as the Gene Engine™, FISH, or optical mapping. Binding of the two-arm probe may also be determined by detecting and measuring cleavage products from the target nucleic acid molecule. In some embodiments, the pattern of binding is indicative of a loss of transcription.

These and other embodiments of the invention will be described in greater detail herein.

### BRIEF DESCRIPTION OF THE DRAWINGS

FIGS. 1A-1D are schematic diagrams showing the different modes of binding and complex formation between a target nucleic acid molecule that is a DNA, and PNA probes of varying types.

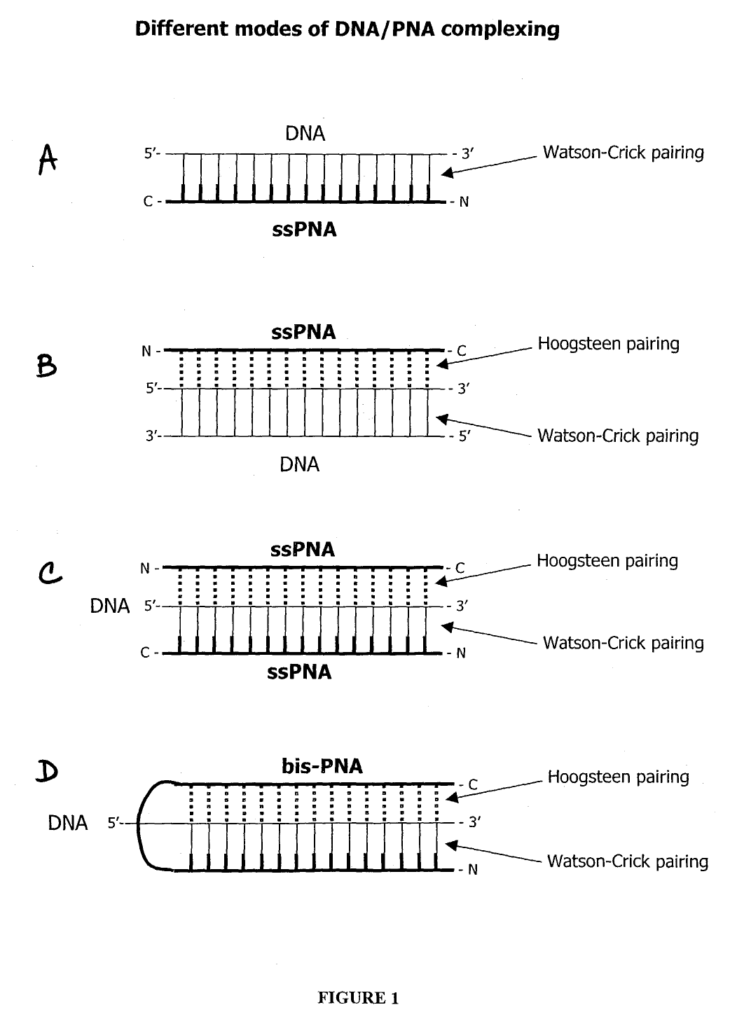

FIG. 2 is a schematic diagram showing the binding of a two-arm PNA to a target dsDNA.

FIG. 3 is a schematic diagram showing the possible structures of a target dsDNA with a two-arm probe.

FIG. 4 is a schematic diagram showing the use of two-arm PNA to protect selected sites against cleavage by for example restriction endonucleases.

It is to be understood that the Figures are provided for illustrative purposes, and they are not required to enable the invention.

### DETAILED DESCRIPTION OF THE INVENTION

The invention relates in part to the discovery of a new probe design that binds to a non-homopurine target with greater efficiency and more rapidly than probes of the prior art. These molecules can be used to bind (and thereby “label”) target nucleic acid molecules. These molecules are referred to as two-arm probes because they are minimally comprised of two strands or arms, one of which forms a Hoogsteen hybrid with a target nucleic acid molecule, and the other which forms a Watson-Crick hybrid with a target nucleic acid molecule. The two-arm probe is designed to bind to different yet adjacent target sites on a target nucleic acid molecule such as a single-stranded or double-stranded nucleic acid. The two-arm probe preferably includes a linker that connects the two arms to each other. The invention provides compositions and methods of use of this two-arm probe.

As used herein, adjacent target sites are sites that are near to each other, but not necessarily immediately next to each other. Contiguous sites are those which are immediately next to each other, as used herein. Thus, as described in greater detail herein, the individual target sites for the Hoogsteen and Watson-Crick arms may be contiguous or they may be spaced apart from each other. Similarly, the target sites may be on the same strand of the target or they may be on opposite strands of a double-stranded target.

The binding efficiency (which may be measured by rate of binding) of the two-arm probe is greater than that of ssPNA or DNA- or RNA-based oligonucleotide probes. However, the two-arm probes of the invention have a more limited set of targets to which they bind (as compared to ssPNA or DNA- or RNA-based oligonucleotide probes) because of the required polypurine sequence of the Hoogsteen arm. While the binding efficiency of a two-arm probe approximates that of a bisPNA, it has a more restricted binding pattern than a bisPNA due to the presence of the Watson-Crick binding arm.

Although not intending to be bound by any particular mechanism, it is believed that in one aspect the invention exploits the ability of the two-arm probe to bind a target nucleic acid molecule in a sequence-specific manner. Once the Hoogsteen binding arm is bound to an appropriate complement, the binding of the Watson-Crick binding arm occurs more efficiently. The Hoogsteen binding arm acts as an anchor holding the Watson-Crick binding arm in the vicinity of its complement on the target nucleic acid molecule.

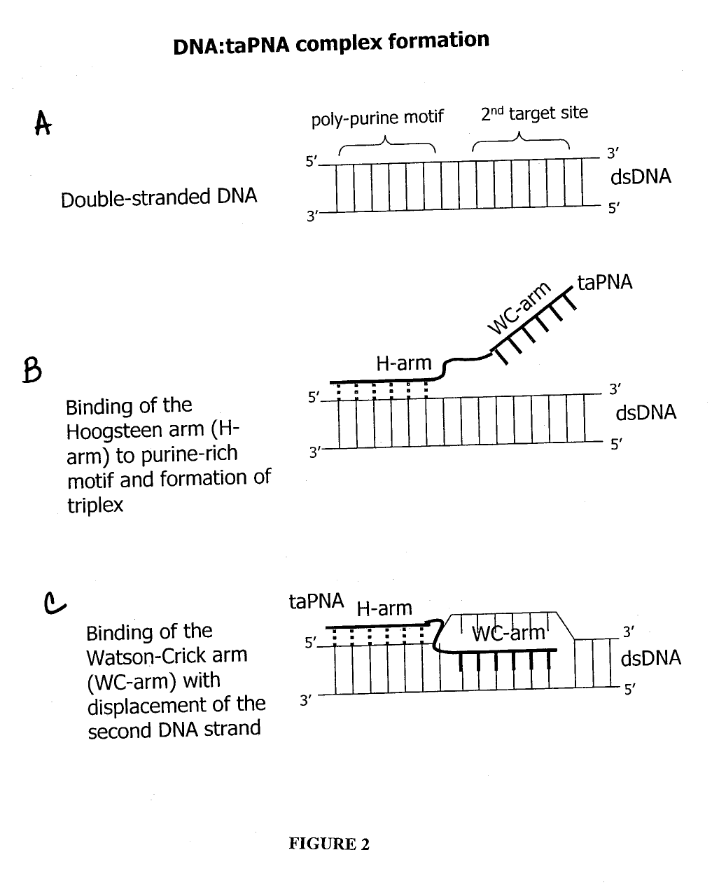

Hybridization of two-arm probe to target nucleic acid molecules can be enhanced using mechanisms similar to those for bisPNA molecules, as described herein. The Hoogsteen binding arm binds directly to a double-stranded helix by Hoogsteen base pairing, and does not require local melting (i.e., opening) and invasion of a double-stranded helix. Hence, the Hoogsteen binding arm can form complexes with double-stranded nucleic acids rapidly because of the low energetic barrier for such binding, and in doing so act as an anchor to position the Watson-Crick binding arm in the vicinity of a target site. Since the Watson-Crick binding arm must invade a double-stranded target, the rate limiting step is local melting of the double-stranded helix. To facilitate opening of the helix, the hybridization reaction is usually performed at elevated temperatures or at lower salt concentrations. To form a hybrid, the Watson-Crick binding arm must be in the vicinity of its target site at the time of melting. Once the local concentration of the Watson-Crick binding arm is increased (via binding of the Hoogsteen binding arm), then the probability that the Watson-Crick binding arm will bind to its target is increased, as shown in FIGS. 2B and 2C.

Hybridization rates of the two-arm probe can also be increased by incorporating positive charges into the two-arm probe structure. An example of this is the incorporation of lysine residues into the PNA structure.

FIG. 2 illustrates the binding of a two-arm probe to a dsDNA target. FIG. 2A shows a dsDNA target with a polypurine motif (that is comprised of either all adenine (A) bases, all guanine (G) bases, or a mixture of A and G bases). FIG. 2B shows the formation of a triplex, comprised of the dsDNA and a Hoogsteen binding arm (the “H-arm”) of the two-arm probe. The Watson-Crick binding arm (i.e., the “WC arm”) has a sequence that is complementary to a nucleotide sequence adjacent (but not necessarily contiguous) to the Hoogsteen binding site. The WC arm, however, cannot hybridize with the target dsDNA until the double-stranded helix opens. FIG. 2C shows that once the dsDNA opens (which can occur, for example, at elevated temperatures), the WC arm of the two-arm probe invades the helix and forms Watson-Crick base pairing with its complementary nucleotide sequence. Note that in this example, the WC arm binds to the opposite DNA strand.

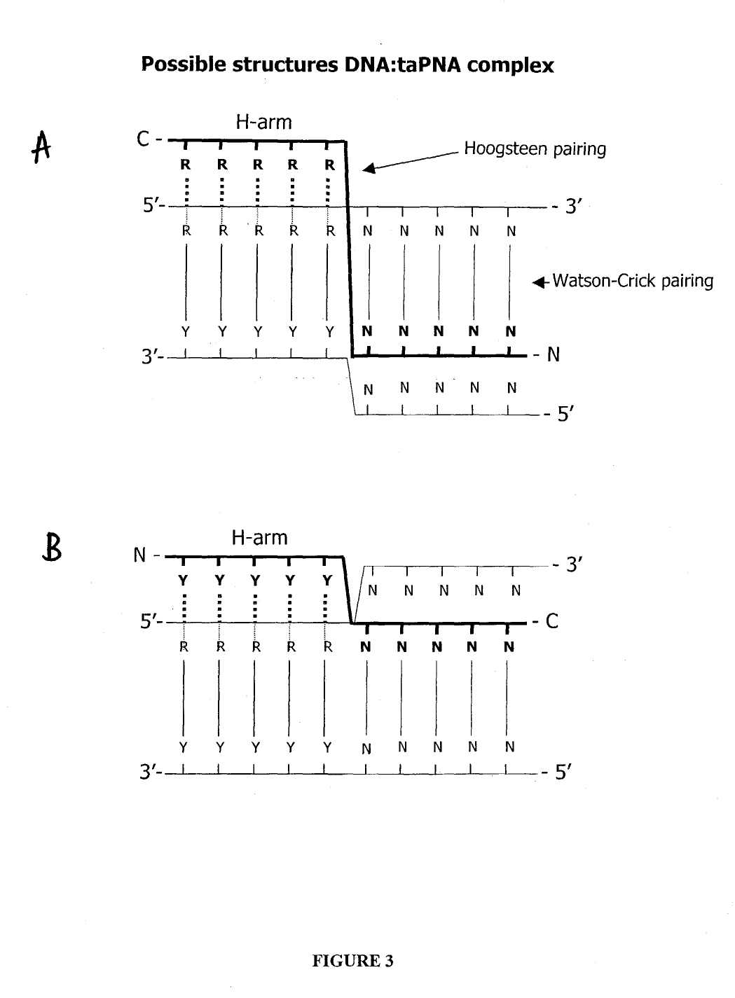

FIG. 3 illustrates the possible orientations of a two-arm probe on a target nucleic acid molecule such as a dsDNA. FIGS. 3A and 3B illustrate orientations of an H arm and a WC arm both of which are PNAs, relative to a target site of a dsDNA. The H arm comprises a polypurine (R) nucleotide sequence (where R can be A, G or a mixture of A and G), and aligns itself with its C-terminus at the 5′-terminus of its target site to form a Hoogsteen-paired complex, as shown in FIG. 3A. Subsequently, the WC arm hydrogen binds to the same strand of DNA to which the H arm is bound, but at a site that is adjacent to the H arm binding site. FIG. 3B illustrates an H arm that comprises a polypyrimidine (Y) nucleotide sequence (where Y can be a cytosine base (C) or a thymine base (T) or a mixture of C and T bases), and aligns itself with its N-terminus at the 5′-terminus of its Hoogsteen target site. The WC arm binds to the opposite strand of DNA via Watson-Crick base pairing. In both cases, the WC arm can bind to a target site consisting of any combination of bases (each N independently may be A, G, C or T, or derivatives or mimics thereof). The WC arm however binds to a sequence that is complementary to itself. The H arm on the other hand may bind to a sequence that is complementary to itself, but it is not so limited. The length of the linker that connects the H and WC arms together will influence the complexes that can be formed and the distance between the individual target sites of each arm. It should be understood that other orientations are also possible, including orientations in which the N-terminus of the two-arm PNA is involved with Watson-Crick binding to one strand of the target and the C-terminus of the two-arm PNA is involved with Hoogsteen binding to the opposite strand of the target. Based on the teachings provided herein, one of ordinary skill will envision the various orientations of Hoogsteen and Watson-Crick bindings that are possible using the two-arm probes of the invention.

In accordance with the invention, two-arm probes have been designed and demonstrated to hybridize with target nucleic acid molecules (such as dsDNA) rapidly and efficiently, particularly as compared to other probe designs. As an example, two-arm probes can form hybrids with dsDNA as rapidly and efficiently as do bisPNA probes of the prior art, which are similarly comprised of two PNAs attached to each other, with or without a linker molecule. One arm of the bisPNA hybridizes to a target nucleic acid molecule by Hoogsteen base pairing, while the other arm hybridizes to the same site on the target nucleic acid molecule by Watson-Crick base pairing. The bisPNA probes are, however, limited in their sequence recognition potential since the Hoogsteen and Watson-Crick binding arms must bind to the same target site. Since Hoogsteen binding can only occur with target homopurine nucleotide sequences, the only sequences that can be detected using bisPNA are homopurine sequences. The two-arm probes provided herein are not limited in this manner, since the Hoogsteen binding arm need not bind to the same target site as the Watson-Crick binding arm (and vice versa).

The target sites for each arm of the two-arm probe are preferably in close proximity (e.g., in the range of 0-1000 base pairs). However, as shown in FIG. 2, they need not be immediately adjacent (i.e., contiguous) to each other (FIG. 2A). In preferred embodiments, the arms of the two-arm probe (and consequently the target sites for the H arm and WC arm) are not immediately adjacent to each other (i.e., they are not contiguous). It is preferable in some instances to separate the H arm and WC arm by a distance of greater than 1000 base pairs (bp), or greater than 500 bp, or greater than 100 bp, or between 1 -100 bp, or between 150 bp, or between 1-25 bp, or between 1-15 bp, or between 3-15 bp, including every integer therebetween as if explicitly recited herein. As described in greater detail below, the two arms of the probe may be conjugated to each other directly, or indirectly via a linker. The distance between the two arms of the two-arm probe (and accordingly, the distance between the target sites to which each arm hybridizes) can be controlled by the length and flexibility of the linker that connects the arms.

The two-arm probe can be used for a number of applications as described herein including but not limited to determining target sequence information and inhibition of transcription and/or translation from a target. Another application is the use of the two-arm probe for sequence-specific termini labeling. The Hoogsteen binding arm will enhance hybridization efficiency, while the Watson-Crick binding arm will bind to target nucleic acid molecule termini and avoid being bound elsewhere on long DNA molecules (e.g., genomic DNA fragments). The ability to perform termini labeling is particularly useful in applications that use single polymer analyzers such as the Gene Engine™ (as described in U.S. Pat. No. 6,355,420 B1, issued Mar. 12, 2002). In these latter applications it is sometimes desirable to label a unique sequence that is located at or near to a terminus of a target molecule (such as a DNA).

The two-arm probes can also be used for detecting the presence (and conversely absence) of particular nucleotide sequences. These sequences may correspond to known mutations associated with particular conditions, or they may be used to identify a source of genetic material (e.g., fingerprinting for forensic or identification purposes). In some embodiments, the sequences are unique, and thus there will be preferably only one two-arm probe bound to a sample. The target sequence may be long, for example a region of genomic or mitochondrial DNA that is amplified or shortened (e.g., as has been observed in Huntington's disease). Alternatively, it may correspond to a single nucleotide polymorphism (SNP).

The binding pattern of the two-arm probes to target nucleic acid molecules can be used to derive sequence information about the targets such as DNA physical maps. As mentioned above, the length of the two-arm probe (and thus its complementary sequence) controls to some extent the resolution of such information. For example, if the two-arm probe is long, then the resolution will be low. The shorter the two arm-probe, the higher the potential resolution will be, provided that contiguously positioned probes can be discerned from each other. That is, the contiguously positioned probes should be spaced at a distance that is greater than the resolution limit of the detection system used. This is described in greater detail in published U.S. Patent Application Publication No. US-2003-0059822-A1, published on Mar. 27, 2003, the entire contents of which are incorporated herein in their entirety.

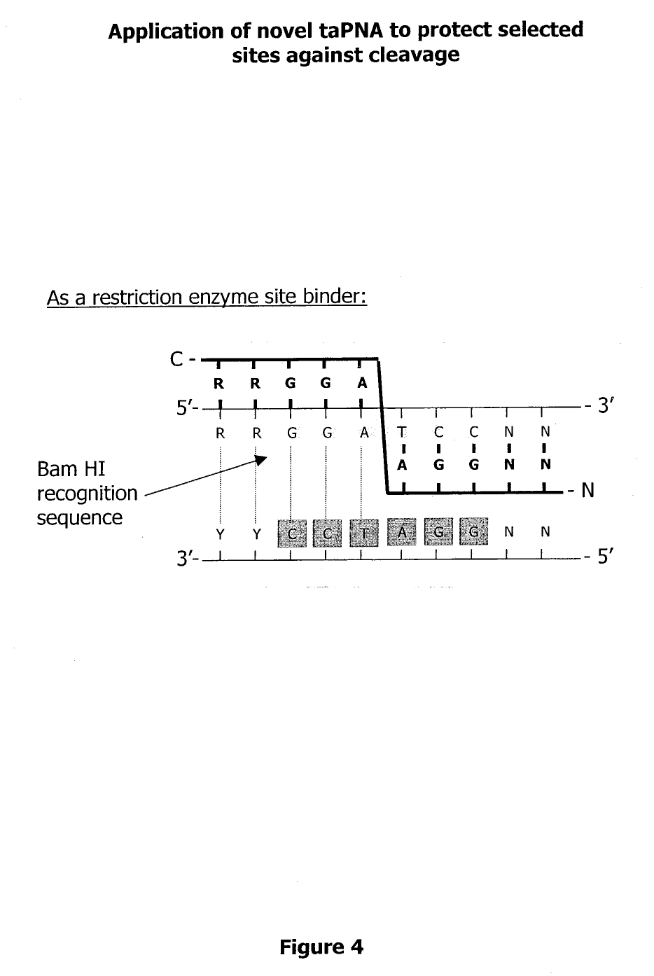

FIG. 4 shows the use of two-arm probes to protect selected sites against cleavage by, for example, restriction endonucleases. Most restriction endonucleases are specific to palindromic sequences (i.e., their ability to cleave a nucleic acid is dependent on their ability to recognize and/or bind to a palindromic sequence). An example of a palindromic sequence is shown in the Figure. The boxed sequence is comprised of a polypyrimidine sequence (i.e., CCT) and a polypurine sequence (i.e., AGG), and accordingly, it can hybridize with the two-arm probes of the invention, and thereby be protected against nuclease attack. The Bam-HI restriction endonuclease recognizes, binds to, and cuts the DNA sequence 5′-GGATCC-3′. This sequence can be hybridized to a two-arm probe, as shown. In some embodiments, it may be preferable to use longer arms that hybridize to the flanking regions of the restriction sequence (e.g., if at room temperature). Complementary flanking sequences can be added onto one or both of the W and H arms.

The Hoogsteen binding arm can be comprised of any type of nucleic acid or nucleic acid mimic, provided that it is capable Hoogsteen hybridization with the target. Its sequence will generally be polypurine or polypyrimidine (as shown in FIGS. 3A and 3B), meaning that it can be comprised of all adenines, all guanines, or a mixture of adenines and guanines, or all cytosines, all thymidines, or a mixture of cytosines and thymidines. In some embodiments, the polypyrimidine nucleotide sequence is preferred for the Hoogsteen binding arm.

The Watson-Crick binding arm similarly can be comprised of any type of nucleic acid or nucleic acid mimic, provided it is capable of Watson-Crick hybridization with the target molecule. Its sequence will be completely random, and dictated only by the particular type of sequence that is sought on the target in a particular application. The two-arm probe (and each of its individual constituent arms) may comprise nucleic acids such as DNA and RNA, as well as nucleic acid mimics such as PNAs (e.g., ssPNA and pcPNA), LNAs, or co-polymers or combinations of the above (e.g., DNA/LNA co-polymer).

In important embodiments, at least one arm, and preferably both arms of the probe are PNAs.

In these latter embodiments, the probe may be referred to as a two-arm PNA. The two-arm probes are comprised of either a polypyrimidine or a polypurine nucleotide sequence that is the Hoogsteen arm, and a random nucleotide sequence that is the Watson-Crick arm.

The lengths of the Hoogsteen and Watson-Crick binding arms are independent of one another, provided that their combined length is sufficient to form a stable complex with a target nucleic acid molecule. The level of hybrid stability required will vary depending upon the application. For example, if the two-arm probe is to be used to label a target for the purpose of in vitro sequencing, then the complex may need to be stable for several hours, possibly at reduced temperatures. If however the two-arm probe is to be used as an anti-sense molecule, to inhibit transcription or translation of a target nucleic acid molecule, then the complex may need to be stable for several days, possibly at body temperatures. The specificity of the probe is dependent in part on its length. The energetic cost of a single mismatch between the two-arm probe and the target nucleic acid molecule is relatively higher for shorter sequences than for longer ones. An equilibrium specificity depends upon the term exp(−ΔG/kT), where ΔG is free energy loss due to the mismatch. Shorter sequences have lower melting temperatures. Near the melting region, the same energy loss can have much stronger effects. A similar mechanism is involved in oligonucleotide hybridization under stringent conditions. Therefore, hybridization of small sequences can be more specific than hybridization of longer sequences.

Another consideration in determining the appropriate probe length is whether the target to be detected is unique or not. If the method is intended to sequence the target nucleic acid molecule, then it will preferable to target non-unique sequences, as this approach will yield more sequence information than will a single binding event corresponding to a unique sequence. Non-unique sequences should be sufficiently spaced apart from each other in the target nucleic acid molecule in order to distinguish contiguous binding events. If the binding events occur within the resolution limit of the detection system, then these events will not be resolved, and thus half the data will be lost. Preferably, the target sequence should occur randomly at distances that can be discerned as separate sites along the target nucleic acid molecule.

The lengths of the two arms may be the same but this is not essential. In some embodiments, it is preferred that the lengths of the Hoogsteen and Watson-Crick binding arms be different. The Hoogsteen binding arm may be as long as the most common length of polypurine or polypyrimidine nucleotide sequences in the target nucleic acid molecule. The Watson-Crick binding arm can be longer or shorter depending, for example, upon the sequence information to be gained. Longer sequences will be more rare, and will be spaced apart at greater distances on average. Shorter sequence will be more common, and will exist at shorter distances to each other. Accordingly, in some instances, shorter Watson-Crick binding arms are desirable if high resolution sequence information is desired. In other instances, longer Watson-Crick binding arms are desirable if unique sequences are sought. It is important to note however that since binding of the two-arm probe involves both arms, the total sequence determines its binding site. Thus, the effect of the WC arm is less than it would be if only the WC arm were present.

Notwithstanding these provisos, the Hoogsteen binding and Watson-Crick binding arms of the invention can be any length ranging from at least 4 nucleotides long to in excess of 1000 nucleotides long. The Hoogsteen binding arm may therefore be 4, 5, 6, 7, 8, 9, 10, 11, 12, 13, 14, 15, 16, 17, 18, 19, 20, at least 20, at least 25, at least 50, at least 75, at least 100, or at least 200 nucleotides in length, or longer. These size ranges apply equally to the Watson-Crick binding arm. Preferred lengths for each of the Hoogsteen and Watson-Crick binding arms are between 5-20, and more preferably are between 5 and 12 nucleotides each.

It should be understood that not all residues of the two-arm probe need hybridize to complementary residues in the target nucleic acid molecule. For example, the target site may be 50 residues in length, yet only 25 of those residues hybridize to the two-arm probe. Preferably, the residues that hybridize are contiguous with each other. Hybridization should however occur at both the Hoogsteen and the Watson-Crick binding arms since the stability of the complex and its binding efficiency are related to the presence of both Hoogsteen and Watson-Crick binding.

In one embodiment, a library of two-arm probes of identical length is generated. The library will preferably contain every possible combination of sequence for that particular length. Each member of a library can be labeled with a distinct label (as discussed below) and is thus discernable from the other library members. A target nucleic acid molecule can be exposed to a library and analyzed for the binding of all two-arm probes that can be detected.

If on the other hand, the method is used to test for the presence of a unique sequence e.g., a mutant sequence such as a translocation event, or a genetic mutation associated with a particular disorder or predisposition to a disorder, then the two-arm probe may be longer in order to capture only its true complement. More than one unique sequence can be analyzed in a given run given the distinct labeling of each two-arm probe, and thus a combination of two-arm probes may be applied to a target nucleic acid molecule and their binding can be analyzed simultaneously, provided that each two-arm probe is uniquely labeled.

It is to be understood that while the Hoogsteen binding arm is used as an anchor to localize the Watson-Crick binding arm, it also imparts sequence information. Since preferably both the Hoogsteen and the Watson-Crick binding arms will be bound to the target at the time sequence information is derived, this information will include the Hoogsteen binding arm sequence (or alternatively, its complement) and the Watson-Crick binding arm sequence (or alternatively, its complement). This is more sequence information than would be available using only the Watson-Crick binding arm.

As stated earlier, the individual target sites of the Hoogsteen and Watson-Crick binding arms need not be immediately adjacent to each other. In fact, in some important embodiments, there is distance between the individual target sites.

The two arms of the probe similarly may be connected to each other with or without a space between them. In some preferred embodiments, there is a distance between the connected ends of the Hoogsteen and Watson-Crick binding arms.

If the length between the Hoogsteen and Watson-Crick binding arms is known, the relative positioning of the target sites will also be known. For example, if the two-arm probe is designed with a distance of 100 Angstroms between the last Hoogsteen base and the first Watson-Crick base (i.e., the distance between the Hoogsteen base connected to the Watson-Crick arm, and vice versa), then there is approximately a 30 base pair distance between the target sites. This distance takes into account a distance of 3.4 Angstroms between two adjacent base pairs in B-form DNA. In cases in which a tether exists between the Watson-Crick and Hoogsteen arms, and if the target sites are on different sides of the helix, an extra 3 nm must be incorporated into the tether region in order to facilitate the placement of the two-arm probe around the DNA cylinder. In the case of a 30 bp distance, both target sites will be on the same side of the DNA helix (given a 10 bp/turn distance) and hence there is no need to incorporate an additional tether length.

As used herein, the “target nucleic acid molecule” is the nucleic acid molecule that is being analyzed or affected using the two-arm probes of the invention. This analysis may involve determining whether a target site is present or absent in a sample, or determining the sequence of the target nucleic acid molecule in part or in its entirety (at varying degrees of resolution), modulating the activity of the target (such as inhibiting transcription from the target, or preventing cleavage of the target), and the like. The two-arm probes can also be used as highly specific PCR primers or probes and/or as molecular beacons.

The two-arm probes are particularly well suited to intracellular applications. For example, there is a limit on the amount of probe that can be added to and taken up by viable cells. There is also a limit on the temperature to which viable cells may be exposed and still remain viable. The compositions of the invention and the methods of use thereof provided herein overcome these limitations due to the accelerated rate of hybridization that can be effected using two-arm probes. Intracellular applications using viable cells include but are not limited to antigene and antisense technology.

The target nucleic acid molecules may be DNA or RNA. The nucleic acid molecules can be directly harvested and/or isolated from a biological sample (such as a tissue or a cell culture) or synthesized de novo. Harvest and isolation of nucleic acid molecules are routinely performed in the art and suitable methods can be found in standard molecular biology textbooks (e.g., such as Maniatis' Handbook of Molecular Biology). Examples of nucleic acid molecules that can be harvested from in vivo sources include genomic DNA, mitochondrial DNA, mRNA, and rRNA, or fragments thereof. The target nucleic acid molecules may be single-stranded and double-stranded nucleic acids. In some embodiments, the target nucleic acid molecules may be comprised of nucleic acid mimics such as PNAs and/or LNAs, but they are not so limited. In important embodiments, the target nucleic acid molecules are DNA or RNA.

The sensitivity of the methods provided herein allows analysis of individual target nucleic acid molecules (i.e., single target nucleic acid molecule analysis). These methods are not dependent upon prior in vitro amplification of a target nucleic acid molecule. Accordingly, in some embodiments, the target nucleic acid molecule is a non in vitro amplified nucleic acid molecule. As used herein, a “non in vitro amplified nucleic acid molecule” refers to a nucleic acid molecule that has not been amplified in vitro using techniques such as polymerase chain reaction or recombinant DNA methods. A non in vitro amplified nucleic acid molecule may be a nucleic acid molecule that is amplified in vivo (in the biological sample from which it was harvested) as a natural consequence of the development of the cells in vivo. This means that the non in vitro nucleic acid molecule may be one that is amplified in vivo as part of locus amplification, a common phenomenon in some mutated or malignant cells. The invention however can be practiced using target nucleic acid molecules that are amplification products, or intermediates thereof, including complementary DNA (cDNA).

The size of the target nucleic acid molecule is not critical to the invention and it is generally only limited by the detection system used. The target nucleic acid molecule can be several nucleotides, several hundred, several thousand, or several million nucleotides in length. In some embodiments, the target nucleic acid molecule may be the length of a chromosome.

The term “nucleic acid molecule” is used herein to mean multiple nucleotides (i.e. molecules comprising a sugar (e.g. ribose or deoxyribose) linked to an exchangeable organic base, which is either a pyrimidine (e.g. cytosine (C), thymine (T) or uracil (U)) or a purine (e.g. adenine (A) or guanine (G)) or an inosine (I), or analogues thereof. “Nucleic acid molecule” and “nucleic acid” are used interchangeably, and refer to oligoribonucleotides as well as oligodeoxyribonucleotides. The terms shall also include polynucleosides (i.e., a polynucleotide minus a phosphate) and any other organic base containing polymer. The organic bases include adenine, uracil, guanine, thymine, cytosine and inosine. Nucleic acid molecules can be naturally occurring (e.g., obtained from natural sources), or synthetic (e.g., made using a nucleic acid synthesizer).

Nucleic acid mimics are also embraced by the invention and include compounds containing bases connected to each other with or without the presence of a sugar and a phosphate backbone. Examples include PNAs and LNAs, but are not so limited.

Nucleic acids and their mimics can include substituted purines and pyrimidines such as C-5 propyne modified bases (Wagner et al., Nature Biotechnology 14:840-844, 1996), 5-methylcytosine, 2-aminopurine, 2-amino-6-chloropurine, 2,6-diaminopurine, hypoxanthine, 2-thiouracil, pseudoisocytosine, and other naturally and non-naturally occurring nucleobases, substituted and unsubstituted aromatic moieties. Other such modifications are well known to those of skill in the art.

The nucleic acid molecules also encompass substitutions or modifications, such as in the bases and/or sugars, and in their backbone compositions. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose.

The Hoogsteen and Watson-Crick binding arms are nucleic acids, derivatives thereof, or nucleic acid mimics. The embodiments recited herein relating to target nucleic acid molecules apply equally to the Hoogsteen and Watson-Crick binding arms of the invention.

The target nucleic acid molecules, and more preferably the two-arm probes, may have a heterogeneous or a homogeneous backbone. When the two-arm probes are used in vivo e.g., added to live cells or tissues containing endo- and exo-nucleases, it may be preferable that they be resistant to degradation from such enzymes. A “stabilized two-arm probe” shall mean a probe that is relatively resistant to in vivo degradation (e.g. via an endo- or exo-nuclease). Examples of stabilized probes are those having a phosphorothioate modified backbone, or a peptide modified backbone (which is inherently non-biodegradable). These examples however are not intended to be limiting.

The target nucleic acid molecules, and more preferably the Hoogsteen binding and Watson-Crick binding arm, can also be stabilized by other backbone modifications. The invention intends to embrace in addition to the peptide and locked nucleic acids discussed herein, the use of the other backbone modifications such as but not limited to phosphorothioate linkages phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine (particularly useful if positive charges are desired in the PNA), and the like. Various PNA modifications are known and tags incorporating such modifications are commercially available from sources such as Boston Probes, Inc., now Applied Biosystems.

As stated above, the two-arm probes can be comprised of various PNA types. PNAs are DNA analogs having their phosphate backbone replaced with 2-aminoethyl glycine residues. These glycine residues are linked to the nucleotide bases through glycine amino nitrogen and methylenecarbonyl linkers. PNAs can bind to both DNA and RNA targets by Watson-Crick or Hoogsteen base pairing, and in so doing form hybrids that are stronger than DNA/DNA or DNA/RNA hybrids.

PNAs can be synthesized from monomers connected by a peptide bond (Nielsen and Egholm 1999), using standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNAs.

PNA has a charge-neutral backbone, and this contributes to its rate of hybridization with DNA-which has a negatively charged backbone (Nielsen and Egholm 1999). The PNA-DNA hybridization rate can be further increased by introducing positive charges in the PNA structure, such as by addition of amino acids with positively charged side chains (e.g., lysines). The stability of a DNA/PNA hybrid is generally independent of the ionic strength of its environment (Orum, et al. 1995), most probably due to the uncharged nature of PNAs. This provides PNAs with the versatility of being used in vivo or in vitro. However, the rate of hybridization of PNAs that comprise positive charges is dependent on ionic strength, and thus is lower in the presence of salt.

The structure of a PNA/DNA hybrid depends on the particular PNA and its sequence. ssPNA binds to ssDNA using Watson-Crick base pairing and preferably in an anti-parallel orientation (i.e., the N-terminus of the ssPNA is opposite the 3′ terminus of the ssDNA). The ssDNA may result from an opening of a dsDNA. The end result of this interaction is a double-stranded complex. ssPNA also can bind to dsDNA with a Hoogsteen base pairing, thereby forming a triple stranded complex (i.e., a triplex) with the dsDNA target (Wittung, et al. 1997).

The presence of mismatches tends to destabilize PNA/DNA hybrids to a greater extent than DNA/DNA hybrids (Egholm, et al. 1993). Accordingly, PNA probes are more specific for a target sequence as they will bind to it in a stable manner only when a high degree of complementarity (or absolute complementarity) exists. This increased specificity can be further enhanced by using shorter PNAs because longer hybrids may be more stable in the presence of a mismatch than will be shorter hybrids.

ssPNA is the simplest of the PNA molecules. This PNA form interacts with nucleic acids to form a hybrid duplex via Watson-Crick base pairing. The duplex has different spatial structure and higher stability than dsDNA (Nielsen and Egholm 1999). However, when different concentration ratios are used and/or in presence of complimentary DNA strand, PNA/DNA/PNA or PNA/DNA/DNA triplexes can also be formed (Wittung, et al. 1997). The formation of duplexes or triplexes additionally depends upon the sequence of the PNA. Thymine-rich homopyrimidine ssPNA forms PNA/DNA/PNA triplexes with dsDNA targets where one PNA strand is involved in Watson-Crick antiparallel pairing and the other is involved in parallel Hoogsteen pairing. Cytosine-rich homopyrimidine ssPNA preferably binds through Hoogsteen pairing to dsDNA forming a PNA/DNA/DNA triplex. If the ssPNA sequence is mixed, it invades the dsDNA target, displaces the DNA strand, and forms a Watson-Crick duplex. Polypurine ssPNA also forms triplex PNA/DNA/PNA with reversed Hoogsteen pairing.

pcPNAs involve two ssPNAs added to dsDNA (Izvolsky, et al. 2000). One pcPNA is complementary to the target sequence, while the other is complementary to the displaced DNA strand. As the PNA/DNA duplex is more stable, the displaced DNA generally does not restore the dsDNA structure. The PNA/PNA duplex is more stable than the DNA/PNA duplex and the PNA components are self-complementary because they are designed against complementary DNA sequences. Hence, the added PNAs preferably hybridize to each other. To prevent the self-hybridization of pcPNA units, modified bases are used for their synthesis including 2,6-diamiopurine (D) instead of adenine and 2-thiouracil (^{S}U) instead of thymine. While D and ^{S}U are still capable of hybridization with T and A respectively, their self-hybridization is sterically prohibited.

pcPNA also makes two base pairings per every nucleotide of the target nucleic acid molecule. Hence, it can bind to short sequences with specificity greater than would be expected from a ssDNA probe. Hybridization of pcPNA can be less efficient than that of bisPNA because it needs three molecules to form the complex.

In some embodiments, two-arm probe that are comprised of PNAs are preferred because PNA/DNA hybrids are more stable than DNA/DNA hybrids. This is important, particularly when analyzing double-stranded nucleic acids such as genomic DNA (especially if performed in situ) because the PNAs will not be displaced by the complementary DNA strand of the target. Accordingly, the PNA/DNA complex can exist for days at room temperature. Moreover, PNAs offer the advantages of efficient and specific hybridization, formation of stable complexes, flexible chemistry, and resistance against degradation by other enzymes.

LNAs form hybrids with DNA, which are at least as stable as PNA/DNA hybrids at low salt concentrations (Braasch and Corey 2001). The energetic barrier for this hybridization however is much higher than that of PNA/DNA hybrids because of the LNA backbone negative charge. Therefore, hybridization kinetics of LNA can be slower than those of PNA. LNA binding efficiency can be increased in some embodiments by adding positive charges to it, as described herein for PNA. Commercial nucleic acid synthesizers and standard phosphoramidite chemistry are used to make LNA oligomers. Therefore, production of mixed LNA/DNA sequences is as simple as that of mixed PNA-peptide sequences.

The two-arm probes are formed by linking the Hoogsteen binding arm to the Watson-Crick binding arm. This linkage can be covalent or non-covalent in nature, although covalent linkage is preferred. The linkage of the Hoogsteen binding arm to the Watson-Crick binding arm should not however interfere with the ability of either arm to recognize and bind to its complementary sequence.

The Hoogsteen binding arm and Watson-Crick binding arm are conjugated to each other either directly or indirectly via a linker. In some instances, a linker can overcome problems arising from steric hindrance, wherein access to the Hoogsteen and/or Watson-Crick target sites is hindered, possibly due to the proximity of the other arm of the two-arm probe. Preferably, the linker is sufficiently long and flexible to allow both arms of the two-arm probe to interact with their respective target sites.

These linkers can be any of a variety of molecules, preferably nonactive, such as straight or even branched carbon chains of C₁-C₃₀, saturated or unsaturated, phospholipids, amino acids, and in particular glycine, and the like, naturally occurring or synthetic. Additional linkers include alkyl and alkenyl carbonates, carbamates, and carbamides. These are all related and may add polar functionality to the linkers such as the C₁-C₃₀ previously mentioned.

A wide variety of spacers can be used, many of which are commercially available, for example, from sources such as Boston Probes, Inc. (now Applied Biosystems). Spacers are not limited to organic spacers, and rather can be inorganic also (e.g., —O—Si—O—, or O—P—O—). Additionally, they can be heterogeneous in nature (e.g., composed of organic and inorganic elements). Essentially, any molecule with reactive groups on it termini can be used as a spacer. Examples include the E linker (which also functions as a solubility enhancer), the X linker which is similar to the E linker, the O linker which is a glycol linker, and the P linker which includes a primary aromatic amino group (all supplied by Boston Probes, Inc., now Applied Biosystems). Other suitable linkers are acetyl linkers, 4-aminobenzoic acid containing linkers, Fmoc linkers, 4-aminobenzoic acid linkers, 8-amino-3, 6-dioxactanoic acid linkers, succinimidyl maleimidyl methyl cyclohexane carboxylate linkers, succinyl linkers, and the like. Another example of a suitable linker is that described by Haralambidis et al. in U.S. Pat. No. 5,525,465, issued on Jun. 11, 1996.

The length of the spacer can vary depending upon the application and the nature of the Hoogsteen binding arm, the Watson-Crick binding arm, and the distance that can be tolerated between their target sites on a target nucleic acid molecule. In some important embodiments, it has a length of not greater than 100 nm, and in some preferred embodiments, it has a length of 1-10 nm.

The conjugations or modifications described herein employ routine chemistry, which is known to those skilled in the art of chemistry. The use of protecting groups and known linkers such as mono- and hetero-bifunctional linkers are documented in the literature (e.g., Herman-Son, 1996) and will not be repeated here.

Specific examples of covalent bonds include those wherein bifunctional cross-linker molecules are used. The cross-linker molecules may be homo-bifunctional or hetero-bifunctional, depending upon the nature of the molecules to be conjugated. Homo-bifunctional cross-linkers have two identical reactive groups. Hetero-bifunctional cross-linkers are defined as having two different reactive groups that allow for sequential conjugation reaction. Various types of commercially available cross-linkers are reactive with one or more of the following groups: primary amines, secondary amines, sulphydryls, carboxyls, carbonyls and carbohydrates. Examples of amine-specific cross-linkers are bis(sulfosuccinimidyl) suberate, bis[2-(succinimidooxycarbonyloxy)ethyl] sulfone, disuccinimidyl suberate, disuccinimidyl tartarate, dimethyl adipimate-2 HCl, dimethyl pimelimidate-2 HCl, dimethyl suberimidate-2 HCl, and ethylene glycolbis-[succinimidyl[succinate]]. Cross-linkers reactive with sulfhydryl groups include bismaleimidohexane, 1,4-di-[3′-(2′-pyridyldithio)-propionamido)] butane, 1 -[p-azidosalicylamido]-4[iodoacetamido] butane, and N-[4-(p-azidosalicylamido) butyl]-3′-[2′-pyridyidithio] propionamide. Cross-linkers preferentially reactive with carbohydrates include azidobenzoyl hydrazine. Cross-linkers preferentially reactive with carboxyl groups include 4-[p-azidosalicylamido] butylamine. Heterobifunctional cross-linkers that react with amines and sulfhydryls include N-succinimidyl-3-[2-pyridyldithio] propionate, succinimidyl [4-iodoacetyl]aminobenzoate, succinimidyl 4-[N-maleimidomethyl] cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl] cyclohexane-1-carboxylate. Heterobifunctional cross-linkers that react with carboxyl and amine groups include 1-ethyl-3-[3-dimethylaminopropyl]-carbodiimide hydrochloride. Heterobifunctional cross-linkers that react with carbohydrates and sulfhydryls include 4-[N-maleimidomethyl]-cyclohexane-1-carboxylhydrazide.2 HCl, 4-(4-N-maleimidophenyl)-butyric acid hydrazide.2 HCl, and 3-[2-pyridyldithio] propionyl hydrazide. The cross-linkers are bis-[β-4-azidosalicylamido)ethyl]disulfide and glutaraldehyde.

Amine or thiol groups may be added at any nucleotide of a synthetic nucleic acid so as to provide a point of attachment for a bifunctional cross-linker molecule. The nucleic acid may be synthesized incorporating conjugation-competent reagents such as Uni-Link AminoModifier, 3′-DMT-C6-Amine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

Noncovalent methods of conjugation may also be used to bind the Hoogsteen binding arm to the Watson-Crick binding arm, or to attach a label to the two-arm probe. Noncovalent conjugation includes hydrophobic interactions, ionic interactions, high affinity interactions such as biotin-avidin and biotin-streptavidin complexation and other affinity interactions. As an example, a molecule such as avidin may be attached to the Hoogsteen binding arm, and its binding partner biotin may be attached to the Watson-Crick binding arm. As another example, avidin may be attached to the two-arm probe (perhaps preferably at the linker if present), and biotin may be attached to an agent.

In some instances, it may be desirable to attach the two arms with using a linker comprising a bond that is cleavable under certain conditions. For example, the bond can be one that cleaves under normal physiological conditions or that can be caused to cleave specifically upon application of a stimulus such as light, whereby one arm can be released, leaving the other arm bound to the target nucleic acid molecule. In some embodiments, it may be desirable to remove the Hoogsteen binding arm, leaving only the Watson-Crick binding arm attached to the target nucleic acid molecule. Readily cleavable bonds include readily hydrolyzable bonds, for example, ester bonds, amide bonds and Schiff's base-type bonds. Bonds which are cleavable by light are known in the art. These cleavable bonds can also be used in linkers that attach the agents or detectable labels to the two-arm probes and/or their constituent arms.

The two-arm probe can be labeled with detectable moieties (i.e., a detectable label). A “detectable label” as used herein is a molecule or compound that can be detected by a variety of methods including fluorescence, electrical conductivity, radioactivity, size, and the like. The label may be of a chemical, peptide or nucleic acid nature although it is not so limited. The label can be detected directly for example by its ability to emit and/or absorb light of a particular wavelength. A label can be detected indirectly by its ability to bind, recruit and, in some cases, cleave another compound which itself may emit or absorb light of a particular wavelength. An example of indirect detection is the use of a first enzyme label which cleaves a substrate into visible products.

The type of label used will depend on a variety of factors, including the nature of the analysis being conducted, the type of the energy source used and the type of target nucleic acid molecule and/or two-arm probe. The label should be sterically chemically compatible with the target nucleic acid molecule and two-arm probe. The label should not interfere with the binding of the two-arm probe to the target nucleic acid molecule, nor should it impact upon the binding specificity of the two-arm probe.

Generally, the detectable label can be selected from the group consisting of an electron spin resonance molecule (such as for example nitroxyl radicals), a fluorescent molecule, a chemiluminescent molecule, a radioisotope, an enzyme substrate, a biotin molecule, an avidin molecule, a streptavidin molecule, a peptide, an electrical charge transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, a nucleic acid, a carbohydrate, an antigen, a hapten, an antibody, an antibody fragment, and a lipid. As used herein, the terms “charge transducing” and “charge transferring” are used interchangeably. The detectable labels described herein are referred to by the systems which detect them. As an example, a chemiluminescent label is a label that can be detected using a chemiluminescent detection system.

Labeling can be carried out either prior to or after two-arm probe formation, or prior to or after binding of the two-arm probe to the target nucleic acid molecule.

Detectable labels include radioactive isotopes such as ³²P or ³H, optical or electron density markers, haptens such as digoxigenin and dintrophenyl, epitope tags such as the FLAG or the HA epitope, and enzyme tags such as alkaline phosphatase, horseradish peroxidase, β-galactosidase, etc. Other labels include chemiluminescent substrates, and fluorophores such as fluorescein isothiocyanate (“FITC”), Texas Red™, tetramethylrhodamine isothiocyanate (“TRITC”), 4, 4-difluoro-4-bora-3a, and 4a-diaza-sindacene (“BODIPY”), Cy-3, Cy-5, Cy-7, Cy-Chrome™, R-phycoerythrin (R-PE), PerCP, allophycocyanin (APC), PharRed™, Mauna Blue, Alexa™ 350, and Cascade Blue®.

Also envisioned by the invention is the use of semiconductor nanocrystals such as quantum dots, described in U.S. Pat. No. 6,207,392 as labels. Quantum dots are commercially available from Quantum Dot Corporation and Evident Technologies.

The two-arm probe and/or target nucleic acid molecules can be labeled using antibodies or antibody fragments and their corresponding antigen or hapten binding partners. Detection of such bound antibodies and proteins or peptides is accomplished by techniques well known to those skilled in the art. Use of hapten conjugates such as digoxigenin or dinitrophenyl is also well suited herein. Antibody/antigen complexes which form in response to hapten conjugates are easily detected by linking a label to the hapten or to antibodies which recognize the hapten and then observing the site of the label. Alternatively, the antibodies can be visualized using secondary antibodies or fragments thereof that are specific for the primary antibody used. Polyclonal and monoclonal antibodies may be used. Antibody fragments include Fab, F(ab)₂, Fd and antibody fragments which include a CDR3 region. The conjugates can also be labeled using dual specificity antibodies.

In some instances, the two-arm probe can be labeled with cytotoxic agents (e.g., antibiotics) or nucleic acid cleaving enzymes. In this way, the two-arm probe can be used for therapeutic purposes as well as for nucleic acid detection and analysis. This may be particularly useful where the two-arm probe has sequence specificity to a known genetic mutation or translocation associated with a disorder or predisposition to a disorder.

The detectable label can be linked or conjugated to the two-arm probe by any means known in the art. For example, the labels may be attached directly to the two-arm probe or attached to a linker which is attached to the two-arm probe. Two-arm probe can be chemically derivatized to include linkers or to facilitate binding to linkers in order to enhance this process. For instance, fluorophores have been directly incorporated into nucleic acids by chemical means but have also been introduced into nucleic acids through active amino or thiol groups introduced into nucleic acids. (Proudnikov and Mirabekov, Nucleic Acid Research, 24:4535-4532, 1996.) An extensive description of modification procedures that can be performed on the two-arm probe, the linker and/or the label can be found in Hermanson, G. T., Bioconjugate Techniques, Academic Press, Inc., San Diego, 1996, which is hereby incorporated by reference.

There are several known methods of direct chemical labeling of DNA (Hermanson, 1996; Roget et al., 1989; Proudnikov and Mirabekov, 1996). One of the methods is based on 10 the introduction of aldehyde groups by partial depurination of DNA. Fluorescent labels with an attached hydrazine group are efficiently coupled with the aldehyde groups and the hydrazine bonds are stabilized by reduction with sodium labeling efficiencies around 60%. The reaction of cytosine with bisulfite in the presence of an excess of an amine fluorophore leads to transamination at the N4 position (Hermanson, 1996). Reaction conditions such as pH, amine fluorophore concentration, and incubation time and temperature affect the yield of products formed. At high concentrations of the amine fluorophore (3M), transamination can approach 100% (Draper and Gold, 1980).

In addition to the above method, it is also possible to synthesize nucleic acids de novo (e.g., using automated nucleic acid synthesizers) using fluorescently labeled nucleotides. Such nucleotides are commercially available from suppliers such as Amersham Pharmacia Biotech, Molecular Probes, and New England Nuclear/Perkin Elmer.

Labels can be attached to the two-arm probe and/or the target nucleic acid molecules or by any mechanism known in the art. For instance, functional groups which are reactive with various labels include, but are not limited to, (functional group: reactive group of light emissive compound) activated ester:amines or anilines; acyl azide:amines or anilines; acyl halide:amines, anilines, alcohols or phenols; acyl nitrile:alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamine:aldehydes or ketones; imido ester:amines or anilines; isocyanate:amines or anilines; and isothiocyanate:amines or anilines.

The labels bound to the two-arm probe may be of the same type, e.g., they may all be fluorescent labels, or they may all be radioactive labels, or they may all be nuclear magnetic labels. Labels that are of the same type are still distinguishable from each other based on the signal they produce once in contact with an energy source (such as for example optical radiation). As an example, two fluorescent labels are distinguishable if they emit fluorescent radiation of different wavelengths. Alternatively, the labels may be of a different type, e.g., one label may be a fluorescent label and one may be a radioactive label.

In one embodiment, the label is a donor or an acceptor fluorophore. A donor fluorophore is a fluorophore which is capable of transferring its fluorescent energy to an acceptor molecule in close proximity. An acceptor fluorophore is a fluorophore that can accept energy from a donor at close proximity. (An acceptor does not have to be a fluorophore. It may be non-fluorescent.) Fluorophores can be photochemically promoted to an excited state, or higher energy level, by irradiating them with light. Excitation wavelengths are generally in the ultraviolet, blue, or green regions of the spectrum. The fluorophores remain in the excited state for a very short period of time before releasing their energy and returning to the ground state. Those fluorophores that dissipate their energy as emitted light are donor fluorophores. The wavelength distribution of the outgoing photons forms the emission spectrum, which peaks at longer wavelengths (lower energies) than the excitation spectrum, but is equally characteristic for a particular fluorophore.

In one variation of an energy transfer system, a combination of fluorescent donor and quenching acceptor is used. In this case, the two-arm probe operates similarly to a “molecular beacon”. When the probe is unbound, the acceptor quenches the fluorescence of the fluorophore due to the linker flexibility. When it is bound, the two arms are separated from each other sufficiently that the acceptor is not able to quench and the probe instead fluoresces.

Analysis of the nucleic acid involves detecting signals from the labels and determining the relative position of those labels relative to one another. In some instances, it may be desirable to further label the target nucleic acid molecule with a standard marker that facilitates comparing the information so obtained with that from other target nucleic acid molecules analyzed. For example, the standard marker may be a backbone label, or a label that binds to a particular sequence of nucleotides (be it a unique sequence or not), or a label that binds to a particular location in the nucleic acid molecule (e.g., an origin of replication, a transcriptional promoter, a centromere, etc.).

One subset of backbone labels are nucleic acid stains that bind nucleic acids in a sequence independent manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc. Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LOPRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, 24, -21, -23, -12, -11, -20, -22, -15, -14, -25 (green), SYTO-81, -80, -82, -83, -84, -85 (orange), SYTO-64, -17, -59, -61, -62, -60, -63 (red).

The nucleic acid molecules are analyzed using linear polymer analysis systems. A linear polymer analysis system is a system that analyzes polymers such as a nucleic acid molecule, in a linear manner (i.e., starting at one location on the polymer and then proceeding linearly in either direction therefrom). As a nucleic acid molecule is analyzed, the detectable labels attached to it are detected in either a sequential or simultaneous manner. When detected simultaneously, the signals usually form an image of the nucleic acid molecule, from which distances between labels can be determined. When detected sequentially, the signals are viewed in histogram (signal intensity vs. time) that can then be translated into a map, with knowledge of the velocity of the nucleic acid molecule. It is to be understood that in some embodiments, the target nucleic acid molecule is attached to a solid support, while in others it is free flowing. In either case, the velocity of the target nucleic acid molecule as it moves past, for example, an interaction station or a detector, will aid in determining the position of the labels relative to each other.

Accordingly, the linear polymer analysis systems are able to deduce not only the total amount of label on a nucleic acid molecule, but perhaps more importantly, the location of such labels. The ability to locate and position the labels allows these patterns to be superimposed on other genetic maps, in order to orient and/or identify the regions of the genome being analyzed. In preferred embodiments, the linear polymer analysis systems are capable of analyzing nucleic acid molecules individually (i.e., they are single molecule detection systems).

An example of such a system is the Gene Engine™ system described in PCT patent applications WO98/35012 and WO00/09757, published on Aug. 13, 1998, and Feb. 24, 2000, respectively, and in issued U.S. Pat. No. 6,355,420 B1, issued Mar. 12, 2002. The contents of these applications and patent, as well as those of other applications and patents, and references cited herein are incorporated by reference in their entirety. This system allows single nucleic acid molecules to be passed through an interaction station in a linear manner, whereby the nucleotides in the nucleic acid molecules are interrogated individually in order to determine whether there is a detectable label conjugated to the nucleic acid molecule. Interrogation involves exposing the nucleic acid molecule to an energy source such as optical radiation of a set wavelength. In response to the energy source exposure, the detectable label on the nucleotide (if one is present) emits a detectable signal. The mechanism for signal emission and detection will depend on the type of label sought to be detected.

The linear polymer analysis system comprises an optical source for emitting optical radiation; an interaction station for receiving the optical radiation and for receiving a nucleic acid molecule that is exposed to the optical radiation to produce detectable signals; and a processor constructed and arranged to analyze the nucleic acid molecule based on the detected radiation including the signals. As described in the above aspect of the invention, the nucleic acid molecule is bound to a two-arm probe.

In one embodiment, the interaction station includes a localized radiation spot. In a further embodiment, the system further comprises a microchannel that is constructed to receive and advance the target nucleic acid molecule through the localized radiation spot, and which optionally may produce the localized radiation spot. In another embodiment, the system further comprises a polarizer, wherein the optical source includes a laser constructed to emit a beam of radiation and the polarizer is arranged to polarize the beam. While laser beams are intrinsically polarized, certain diode lasers would benefit from the use of a polarizer. In some embodiments, the localized radiation spot is produced using a slit located in the interaction station. The slit may have a slit width in the range of 1 nm to 500 nm, or in the range of 10 nm to 100 nm. In some embodiments, the polarizer is arranged to polarize the beam prior to reaching the slit. In other embodiments, the polarizer is arranged to polarize the beam in parallel to the width of the slit.

In yet another embodiment, the optical source is a light source integrated on a chip. Excitation light may also be delivered using an external fiber or an integrated light guide. In the latter instance, the system would further comprise a secondary light source from an external laser that is delivered to the chip.

Another method for analyzing a target nucleic acid molecule comprises generating optical radiation of a known wavelength to produce a localized radiation spot; passing a target nucleic acid molecule through a microchannel; irradiating the target nucleic acid molecule at the localized radiation spot; sequentially detecting radiation resulting from interaction of the target nucleic acid molecule with the optical radiation at the localized radiation spot; and analyzing the target nucleic acid molecule based on the detected radiation.

In one embodiment, the method further employs an electric field to pass the target nucleic acid molecule through the microchannel. In another embodiment, detecting includes collecting the signals over time while the target nucleic acid molecule is passing through the microchannel.

Other single molecule nucleic acid analytical methods which involve elongation of a target nucleic acid molecule, such as a DNA molecule, can also be used in the methods of the invention. These include optical mapping (Schwartz et al., 1993; Meng et al., 1995; Jing et al., 1998; Aston, 1999) and fiber-fluorescence in situ hybridization (fiber-FISH) (Bensimon et al., 1997). In optical mapping, nucleic acid molecules are elongated in a fluid sample and fixed in the elongated conformation in a gel or on a surface. Restriction digestions are then performed on the elongated and fixed nucleic acid molecules. Ordered restriction maps are then generated by determining the size of the restriction fragments. In fiber-FISH, nucleic acid molecules are elongated and fixed on a surface by molecular combing. Hybridization with fluorescently labeled two-arm probe allows determination of sequence landmarks on the target nucleic acid molecules. Both methods require fixation of elongated molecules so that molecular lengths and/or distances between markers can be measured. Pulse field gel electrophoresis can also be used to analyze the labeled nucleic acid molecules. Pulse field gel electrophoresis is described by Schwartz et al. (1984). Other nucleic acid analysis systems are described by Otobe et al. (2001), Bensimon et al. in U.S. Pat. No. 6,248,537, issued Jun. 19, 2001, Herrick and Bensimon (1999), Schwartz in U.S. Pat. No. 6,150,089 issued Nov. 21, 2000 and U.S. Pat. No. 6,294,136, issued Sep. 25, 2001. Other linear polymer analysis systems can also be used, and the invention is not intended to be limited to solely those listed herein.

The systems described herein will encompass at least one detection system. The nature of such detection systems will depend upon the nature of the detectable label. The detection system can be selected from any number of detection systems known in the art. These include an electron spin resonance (ESR) detection system, a charge coupled device (CCD) detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system, many of which are electromagnetic detection systems.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application are incorporated by reference herein in their entirety.

We claim:

1. A composition comprising
a Hoogsteen binding arm that binds by Hoogsteen base pairing to a target nucleic acid molecule at a first target site, and
a Watson-Crick binding arm that binds by Watson-Crick base pairing to the target nucleic acid molecule at a second target site,
wherein the Hoogsteen binding arm and the Watson-Crick binding arm are conjugated to each other, and are comprised of nucleic acid or nucleic acid mimic elements.

2. The composition of claim 1, wherein the Hoogsteen binding arm is selected from the group consisting of a DNA, an RNA, a PNA, and an LNA.

3. The composition of claim 1, wherein the Watson-Crick binding arm is selected from the group consisting of a DNA, an RNA, a PNA, and an LNA.

4. The composition of claim 1, wherein the target nucleic acid molecule is a DNA or an RNA.

5. The composition of claim 1, wherein the Hoogsteen binding arm has at least one backbone modification.

6. The composition of claim 1, wherein the Watson-Crick binding arm has at least one backbone modification.

7. The composition of claim 5 or 6, wherein the at least one backbone modification is selected from the group consisting of a peptide modification, and a phosphorothioate modification.

8. The composition of claim 1, wherein the Hoogsteen binding arm and the Watson-Crick binding arm are conjugated to each other covalently.

9. The composition of claim 1, wherein the Hoogsteen binding arm and the Watson-Crick binding arm are conjugated to each other using a linker molecule.

10. The composition of claim 9, wherein the linker molecule is selected from the group consisting of 8-amino-3,6-dioxaoctanoic acid (O-linker), E-linker, and X-linker.

11. The composition of claim 9, wherein the linker molecule comprises a cleavable bond.

12. The composition of claim 9, wherein the linker molecule has a length of less than 100 Angstroms.

13. The composition of claim 1, wherein the Hoogsteen binding arm has a nucleotide sequence that is a homopurine nucleotide sequence or homopyrimidine nucleotide sequence.

14. The composition of claim 1, wherein the Watson-Crick binding arm has a nucleotide sequence that is random.

15. The composition of claim 1, wherein the Hoogsteen binding arm is 5-12 nucleotides in length.

16. The composition of claim 1, wherein the Watson-Crick binding arm is 5-12 nucleotides in length.

17. The composition of claim 1, wherein the Hoogsteen binding arm and the Watson-Crick binding arm have different lengths.

18. The composition of claim 1; wherein the first target site and the second target site are spaced apart from each other by a distance selected from the group consisting of 1 base pair, 2 base pairs, 5 base pairs, 7 base pairs, 10 base pairs, 20 base pairs, and 25 base pairs.

19. The composition of claim 1, wherein the Hoogsteen binding arm and the Watson-Crick binding arm, when both are bound to their respective target sites, are spaced apart from each other by a distance selected from the group consisting of 1 base pair, 2 base pairs, 5 base pairs, 7 base pairs, 10 base pairs, 20 base pairs, and 25 base pairs.

20. The composition of claim 1, wherein the Hoogsteen binding arm is conjugated to an agent.

21. The composition of claim 1 or 20, wherein the Watson-Crick binding arm is conjugated to an agent.

22. The composition of claim 20 or 21, wherein the agent is a detectable label.

23. The composition of claim 22, wherein the detectable label is selected from the group consisting of an electron spin resonance molecule (e.g., nitroxyl radicals), a fluorescent molecule, a chemiluminescent molecule, a radioisotope, an enzyme substrate, a biotin molecule, an avidin molecule, an electrical charge transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, a nucleic acid, a carbohydrate, an antigen, a hapten, an antibody, an antibody fragment, and a lipid.

24. The composition of claim 22, wherein the detectable label is detected using a detection system selected from the group consisting of a charge coupled device detection system, an electron spin resonance detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system.

25. The composition of claim 20 or 21, wherein the agent is a cytotoxic agent.

26. The composition of claim 1, wherein the target nucleic acid molecule is a genomic DNA molecule or a mitochondrial DNA molecule.

27. A composition comprising
a Hoogsteen binding arm that binds by Hoogsteen base pairing to a target nucleic acid molecule at a first target site, and
a Watson-Crick binding arm that binds by Watson-Crick base pairing to the target nucleic acid molecule at a second target site
wherein the Hoogsteen binding arm and the Watson-Crick binding arm are conjugated to each other through a linker.

28. A method for labeling a target nucleic acid molecule comprising
a) contacting the target nucleic acid molecule with a composition of claim 1 or 27, and
b) allowing the composition to bind specifically to the target nucleic acid molecule.

29. The method of claim 28, further comprising detecting binding of the composition to the target nucleic acid molecule.

30. The method of claim 28, wherein the Hoogsteen binding arm is selected from the group consisting of a DNA, an RNA, a PNA, and an LNA.

31. The method of claim 28, wherein the Watson-Crick binding arm is selected from the group consisting of a DNA, an RNA, a PNA, and an LNA.

32. The method of claim 28, wherein the Hoogsteen binding arm has at least one backbone modification.

33. The method of claim 28, wherein the Watson-Crick binding arm has at least one backbone modification.

34. The method of claim 32 or 33, wherein the at least one backbone modification is selected from the group consisting of a peptide modification and a phosphorothioate modification.

35. The method of claim 28, wherein the Hoogsteen binding arm and Hoogsteen binding arm are conjugated to each other covalently.

36. The method of claim 28, wherein the Hoogsteen binding arm and Hoogsteen binding arm are conjugated to each other using a linker molecule.

37. The method of claim 36, wherein the linker molecule is selected from the group consisting of 8-amino-3,6-dioxaoctanoic acid (O-linker), E-linker, and X-linker.

38. The method of claim 36, wherein the linker molecule comprises a hydrolyzable cleavable.

39. The method of claim 36, wherein the linker molecule has a length of less than 100 Angstroms.

40. The method of claim 28, wherein the Hoogsteen binding arm has a nucleotide sequence that is a homopurine nucleotide sequence or homopyrimidine nucleotide sequence.

41. The method of claim 28, wherein the Watson-Crick binding arm has a nucleotide sequence that is random.

42. The method of claim 28, wherein the Hoogsteen binding arm is 5-12 nucleotides in length.

43. The method of claim 28, wherein the Watson-Crick binding arm is 5-12 nucleotides in length.

44. The method of claim 28, wherein the Hoogsteen binding arm and the Watson-Crick binding arm have different lengths.

45. The method of claim 28, wherein the first target site and the second target site are spaced apart from each other by a distance selected from the group consisting of 1 base pair, 2 base pairs, 5 base pairs, 7 base pairs, 10 base pairs, 20 base pairs, and 25 base pairs.

46. The method of claim 28, wherein the Hoogsteen binding arm and the Watson-Crick binding arm, when both are bound to their respective target sites, are spaced apart from each other by a distance selected from the group consisting of 1 base pair, 2 base pairs, 5 base pairs, 7 base pairs, 10 base pairs, 20 base pairs, and 25 base pairs.

47. The method of claim 28, wherein the Hoogsteen binding arm is conjugated to an agent.

48. The method of claim 28 or 47, wherein the Watson-Crick binding arm is conjugated to an agent.

49. The method of claim 47 or 48, wherein the agent is a detectable label.

50. The method of claim 49, wherein the detectable label is selected from the group consisting of an electron spin resonance molecule (e.g., nitroxyl radicals), a fluorescent molecule, a chemiluminescent molecule, a radioisotope, an enzyme substrate, a biotin molecule, an avidin molecule, an electrical charge transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, a nucleic acid, a carbohydrate, an antigen, a hapten, an antibody, an antibody fragment, and a lipid.

51. The method of claim 49, wherein the detectable label is detected using a detection system selected from the group consisting of a charge coupled device detection system, an electron spin resonance detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system.

52. The method of claim 47 or 48, wherein the agent is a cytotoxic agent.

53. The method of claim 48, wherein the agent is a nucleic acid cleaving agent.

54. The method of claim 28, wherein the target nucleic acid molecule is a DNA or an RNA molecule.

55. The method of claim 28, wherein the target nucleic acid molecule is a genomic DNA molecule or a mitochondrial DNA molecule.

56. The method of claim 29, further comprising determining a pattern of binding of the composition to the target nucleic acid molecule.

57. The method of claim 56, wherein the pattern of binding is determined using a linear polymer analysis system, FISH, or optical mapping.

58. The method of claim 56, wherein the pattern of binding is determined by detecting and measuring cleavage products from the target nucleic acid molecule.

59. The method of claim 56, wherein the pattern of binding is indicative of a loss of transcription.

60. The composition of claim 1, wherein the Hoogsteen binding arm comprises a PNA.

61. The composition of claim 1 or claim 60, wherein the Watson-Crick binding arm comprises a PNA.

62. The method of claim 28, wherein the Hoogsteen binding arm comprises a PNA.

63. The method of claim 28, wherein the Watson-Crick binding arm comprises a PNA.
